(19) United States
(12) Patent Application Publication
(10) Pub. No.: US 2012/0148517 A1
(43) Pub. Date: Jun. 14, 2012

(54) Hair Relaxing and Straightening Compositions

(75) Inventors: Douglas GILMORE, Cave Creek, AZ (US); Peter LENTINI, West Babylon, NY (US); Tiffany OLIPHANT, Chandler, AZ (US); James H. BROWN, Scottsdale, AZ (US); David A. ASHLEY, Mesa, AZ (US)

(73) Assignee: International Flora Technologies, Ltd., Chandler, AZ (US)

(21) Appl. No.: 13/312,736

(22) Filed: Dec. 6, 2011

Related U.S. Application Data
(60) Provisional application No. 61/420,116, filed on Dec. 6, 2010.
(60) Provisional application No. 61/448,341, filed on Mar. 2, 2011.
(60) Provisional application No. 61/508,471, filed on Jul. 15, 2011.

(51) Int. Cl. A61K 8/92; A61Q 5/04

(52) U.S. Cl. 424/70.4; 424/70.2

(57) ABSTRACT

The inventors disclose hair relaxing and/or straightening compositions, methods of making hair relaxing compositions, kits containing hair relaxing compositions, methods of straightening hair with hair relaxing compositions, and hair relaxed and/or straightened by hair relaxing compositions and/or methods.

### PRIORITY CLAIMS

This Application claims the benefit of U.S. Provisional Patent Application Ser. No. 61/420,116, which was filed on 6 Dec. 2010 and is incorporated herein by this reference in its entirety. This Application also claims the benefit of U.S. Provisional Patent Application Ser. No. 61/448,341, which was filed on 2 Mar. 2011 and is incorporated herein by this reference in its entirety. This Application also claims the benefit of U.S. Provisional Patent Application Ser. No. 61/508,471, which was filed on 15 Jul. 2011 and is incorporated herein by this reference in its entirety.

### TECHNICAL FIELD OF THE INVENTION

The following discussion generally pertains to the fields of cosmetology and personal care. More specifically, the following disclosure relates to compositions for relaxing and/or straightening hair, kits containing such compositions, methods of making such compositions, and methods of using such compositions for the relaxation and/or straightening of hair.

### SUMMARY OF INVENTION

The compositions, methods of making, kits and hair treatment methods provided are representative and exemplary and are not intended to limit the scope of the inventive embodiments herein claimed or otherwise described.

In an exemplary embodiment, a representative composition comprises water, a hydrolysate of an oil and/or wax, an alcohol, and a buffer substance. The hydrolysate of the oil and/or wax is comprised of about 6-95% by weight of organic unsaponifiable materials and about 5-96% by weight of organic saponifiable materials. The composition has a pH ranging from about 10 to about 14 when measured in an aqueous solution of 10% by weight of the composition and 90% by weight water. The composition may further comprise a hydroxide salt.

In another representative embodiment, a composition made by a process is disclosed. The composition may be made by a process comprising the steps of combining, in any order, water, an alcohol, a buffer substance, and an oil and/or wax hydrolysate to form a mixture, and adjusting the pH of the mixture to give a pH range from about 10 to about 14 when measured in an aqueous solution of 10% by weight of the composition and 90% by weight water. The oil and/or wax hydrolysate is comprised of a hydrolysate of an oil and/or wax having about 6-95% by weight of organic unsaponifiable materials and about 5-94% by weight of organic saponifiable materials. The pH of the composition may be adjusted, for example, without limitation, by addition of an organic and/or an inorganic hydroxide salt to the mixture.

In an additional representative embodiment, a kit containing a composition is disclosed. The kit may contain instructions for using the composition or any other application aids, whether now known or hereafter devised by those skilled in the art. Instructions may direct that the composition be used to relax and/or straighten hair. The instructions may be reduced to any tangible medium of expression, and may employ various languages, for example, but not limited to, English, German, Spanish, French, Italian, Portuguese, Japanese, Korean, Russian, and/or Chinese.

In a further representative embodiment, a method for making a composition is disclosed. The method comprises the steps of combining, in any order, water, an alcohol, a buffer substance, and an oil and/or wax hydrolysate to form a mixture, and adjusting the pH of the mixture to give a pH range from about 10 to about 14 when measured in an aqueous solution of 10% by weight of the composition and 90% by weight water. The oil and/or wax hydrolysate may be a hydrolysate of an oil and/or wax comprising about 6-95%o by weight of organic unsaponifiable materials and about 5-94% by weight of organic saponifiable materials. The pH of the composition may be adjusted by addition of an organic and/or inorganic hydroxide salt to the mixture. The method may additionally employ the application of heat.

In another representative embodiment, a method of straightening hair is disclosed.

The method comprises the step of applying a composition to the hair in an amount and for a time sufficient to relax and/or straighten the hair. The hair may optionally be in need of or otherwise disposed to straightening and/or relaxation. The hair may optionally be human hair.

In yet another representative embodiment, hair straightened and/or relaxed by a composition and/or method is disclosed. The hair itself may be comprised or otherwise intended for use in, for example, without limitation: a wig, a hair piece, a toupee, a tress and/or a hair extension. The hair may optionally be in need of or otherwise disposed to relaxation and/or straightening. The hair may optionally be human hair.

### DESCRIPTION OF DRAWINGS

Exemplary embodiments will hereinafter be described in conjunction with the following drawing figures, wherein:

FIG. 1 generally depicts a group of hair tresses that were treated with hair relaxer/straightening formulations in accordance with representative embodiments of the present invention. The tresses were treated with various concentrations of relaxer/straightening compositions for various periods of time corresponding to the following, in order from left to right: 50% strength for 30 minutes, 50% strength for 45 minutes, 50% strength for 60 minutes, 25% strength for 30 minutes, 25% strength for 45 minutes, 25% strength for 60 minutes, 10% strength for 30 minutes, 10% strength for 45 minutes, and 10% strength for 60 minutes. For the purposes of references made to FIG. 1, “% strength” indicates the fractional weight concentration of a relaxer composition in accordance with an exemplary embodiment of the present invention as contained in a finished relaxer formulation. By way of analogy, see the “FC3” relaxer composition, for example, as indicated in Table 1 and its inclusion in the relaxer formulations described in Table 2 (at 16.7% strength) and Table 3 (at 25% strength) vide infra.

FIG. 2 generally depicts a group of hair tresses that were treated with hair relaxer/straightening formulations in accordance with representative embodiments of the present invention. The tresses were treated with increasing concentrations of relaxer/straightening compositions for thirty (30) minutes each corresponding to the following, in order from left to right: 10%, 25% and 50%. For the purposes of references made to FIG. 2, “% strength” indicates the fractional weight concentration of a relaxer composition in accordance with an exemplary embodiment of the present invention as contained in a finished relaxer formulation. By way of analogy, see the “FC3” relaxer composition, for example, as indicated in Table 1 and its inclusion in the relaxer formulations described in Table 2 (at 16.7% strength) and Table 3 (at 25% strength) vide infra.

FIG. 3 generally depicts an untreated control hair tress on the left and a hair tress that was treated with a hair relaxer/straightening formulation in accordance with a representative embodiment of the present invention. The treated tress (on the right) was representatively treated with a fall strength (100%) concentration of relaxer/straightening composition for thirty (30) minutes. For the purposes of references made to FIG. 3, “% strength” indicates the fractional weight concentration of a relaxer composition in accordance with an exemplary embodiment of the present invention. By way of analogy, see the “FC3” relaxer composition, for example, as indicated in Table 1 vide infra.

Definitions

All of the various aspects, embodiments and options disclosed herein can be combined in any and all variations or permutations.

As used herein, “a” means one or more, unless otherwise specified.

Open terms such as “include”, “includes”, “including”, “contain”, “contains”, “containing”, “have”, “has”, “having”, and/or the like, mean “comprising”,

The terms “may” and “can” are both intended to equivalently reference permissive constructions, and neither are intended to suggest a degree of permittivity attributed to one as being greater than the other.

The term “or” is intended to denote conjunctive or disjunctive alternatives. The term “or” is not intended to suggest exclusivity among or between any of the member elements or combination of member elements so referenced in a list or set.

The term “and” is intended to denote conjunctive or sequential disposition in time, place, configuration, aggregation or other arrangement. The term “and” is not intended to suggest any required order to be established or maintained among or between any of the member elements or combination of member elements so referenced in a list or set.

Some inventive embodiments contemplate numerical ranges. Every numerical range provided herein includes the range endpoints as discrete inventive embodiments. When a numerical range is provided, all individual values and sub-ranges therein are understood to be present as if explicitly written out. For example, the range 10-50 is understood to include the sub-range 20-40 as if it were explicitly written out.

When used to modify a discrete number, the term “about” means ±10% of the number it modifies. For example, “about 90” provides for a range of values from 81 to 99, including the number 90. When the term “about” is used to modify a range, “about” means ±10% of the larger number in the range as applied against both the lower endpoint and the upper endpoint. For example, “about 49-80” provides for a range of from 41 (i.e., 49 minus 8) to 88 (i.e., 80 plus 8). For the purposes herein, in no event will the amount of the modification as calculated from the upper endpoint of a range be applied against the lower endpoint value of the range to effectively reduce the lower endpoint to a value less than zero. For example, “about 5-70” provides for a range of from 0 to 77.

When the claims and/or specification reference a composition having a percentage (%) weight fraction of a specific component (e.g., organic saponifiable materials, organic unsaponifiable materials, etc.), the “weight fraction” is intended to reference the explicitly described component so constituted before any other materials are added or taken away. Accordingly, it is the intent of the inventors to prevent the avoidance of literal or equivalent infringement hereunder by operation of physical dilution or effective concentration (subsequent or ab initio) in attempts to design around the values and/or ranges recited herein. For example, “a hydrolysate of an oil and/or wax, said hydrolysate comprising about 6-90% by weight of organic unsaponifiable materials and about 5-95% by weight of organic saponifiable materials” shall be understood to have the recited weight percentage values reference the explicitly described “hydrolysate” component and not the effective percentages otherwise resulting from pre-/post-addition of alcohol, buffer substance, water, solvents, emulsifiers, fragrances, pigments, dyes, and/or the like, etc.

A buffer substance contains and/or is an acid, a base, and/or a salt. In one representative embodiment, the buffer substance may be a mixture of an acid and its conjugate base. The buffer substance may, but is not required to, buffer any composition to which it is added, and may be, but need not be, part of a buffer system.

To relax hair means to decrease the amount of curl in the hair (e.g., to partially or fully remove curl from the hair). When decreases in the amount of hair curl are described, they are relative to control hair that has not been treated with a hair relaxing process and/or a hair relaxing composition. Assessments of hair straightening generally correspond to a subjective metric employed to indicate 100% as substantially complete straightening of the hair relative to an un-treated control (itself referenced as 0%). Accordingly, a score of 50% relaxation would correspond to a subjective assessment where the hair so scored would be observed to have only half (50%) of the curl as compared with an un-treated control.

Partial relaxation of the hair may be understood to alternatively, conjunctively and/or sequentially comprise smoothening or softening of larger waves/curls and/or defrizzing of smaller waves/curls.

The concept of a “hydrolysate of an oil and/or wax” shall be understood to mean a “hydrolysate of an oil and/or hydrolysate of a wax”. The concept of an “oil and/or wax hydrolysate” shall be understood to mean an “oil hydrolysate and/or wax hydrolysate”, The phrases “hydrolysate of an oil and/or hydrolysate of a wax” and “oil hydrolysate and/or wax hydrolysate” shall be understood to have the same meaning.

Representative hydrolysates may be produced in situ and/or via chemical hydrolysis. Hydrolysates in accordance with various representative embodiments of the invention may also be at least partial produced via a combination of two or more chemical components that otherwise would correspond to the products produced from one or more hydrolysis reactions. Hydrolysates in accordance with various other representative embodiments of the present invention may correspond to at least one substantially synthetic component that is suitably adapted to mimic or otherwise at least partially function similar to one or more chemical components corresponding to the products produced from one or more hydrolysis reactions.

Where any reference herein is made indicating use of the disclosed invention to straighten/relax/smoothen/soften human hair, it is understood that the hair of any mammalian animal my also be so treated to accomplish any substantially similar goal of straightening/relaxing/smoothening/softening the hair of a mammalian subject.

Terms in this application control in the event of a conflict with a patent, patent application, or publication term that is incorporated by reference.

### DETAILED DESCRIPTION OF THE INVENTION

People routinely relax and/or straighten their hair for cosmetic and aesthetic reasons. Conventional hair relaxing methods and compositions employ extreme temperatures and/or chemicals that can damage hair, are hazardous, and/or produce or are themselves responsible for malodor. Furthermore, conventional hair straightening methods and compositions can relax hair for a relatively brief duration of time and so require regular, repeated applications. There is a need for hair relaxing compositions, kits and methods: which do not need to employ, for example, extreme physical measures such as heating hair at high temperature; that do not need to employ hazardous substances such as, for example, formaldehyde; that do not need to employ offensive, malodorous substances such as, for example, thiols; and that relax hair for a desired duration of time. Finally, there is a need for relaxed and/or straightened hair that is suitably adapted for use, for example, in a wig, a hair piece, a hair extension, a hair tress, or a toupee.

Disclosed herein are compositions, methods and kits for relaxing and/or straightening hair, and hair that has been relaxed and/or straightened by the disclosed methods and/or compositions, that can address the above-described needs and problems.

In a representative embodiment, a composition is disclosed. The composition comprises water, a hydrolysate of an oil and/or wax, an alcohol, and a buffer substance. The hydrolysate of the oil and/or wax comprises about 6-95% by weight of organic unsaponifiable materials and about 5-96% by weight of organic saponifiable materials. The composition has a pH ranging from about 10 to about 14 when measured in an aqueous solution of 10% by weight of the composition and 90% by weight water. The composition may further comprise a hydroxide salt.

pH

The pH of the composition can be measured, for example, in an aqueous solution of 10% by weight of the composition and 90% by weight water. The pH of the composition can be, for example, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, about 13.0, about 13.1, about 13.2, about 13.3, about 13.4, about 13.5, about 13.6, about 13.7, about 13.8, or about 13.9.

The pH of the composition can be, for example, at least 10, at least 10.1, at least 10.2, at least 10.3, at least 10.4, at least 10.5, at least 10.6, at least 10.7, at least 10.8, at least 10.9, at least 11.0, at least 11.1, at least 11.2, at least 11.3, at least 11.4, at least 11.5, at least 11.6, at least 11.7, at least 11.8, at least 11.9, at least 12.0, at least 12.1, at least 12.2, at least 12.3, at least 12.4, at least 12.5, at least 12.6, at least, 12.7, at least 12.8, at least 12.9, at least 13.0, at least 13.1, at least 13.2, at least 13.3, at least 13.4, at least 13.5, at least 13.6, at least 13.7, at least 13.8, or at least 13.9.

The pH of the composition can range, for example from about 10 to about 10.5, from about 10 to about 11, from about 10 to about 11.5, from about 10 to about 12.0, from about 10 to about 12.5, from about 10 to about 13.0, from about 10 to about 13.5, from about 10.5 to 14, from about 11.0 to 14, from about 11.5 to 14, from about 12.0 to 14, from about 12.5 to 14, from about 13.0 to 14, or from about 13.5 to 14.

Hydrolysates

Hydrolysates of the oil and/or wax may comprise, for example, from about 6-90%, from about 10-85%, from about 20-75%, from about 30-70%, from about 40-65%, from about 50-60%, from about 6-10%, from about 6-15%, from about 6-20%, from about 6-25%, from about 6-30%, from about 6-35%, from about 6-40%, from about 50-90%, from about 55-90%, from about 60-90%, from about 65-90%, from about 70-90%, from about 75-90%, from about 80-90%, or from about 85-90% by weight, based on the weight of the hydrolysate, of organic unsaponifiable materials.

Representative hydrolysates of the oil and/or wax may comprise, for example, about 6%, about 10%, about 15%, about 20%, about 25%, about 30%, about 35%, about 40%, about 45%, about 50%, about 55%, about 60%, about 65%, about 70%, about 75%, about 80%, about 85%, or about 90% by weight, based on the weight of the hydrolysate, of organic unsaponifiable materials.

Representative hydrolysates of the oil and/or the wax can comprise, for example, from about 5-95%, from about 10-90%, from about 15-85%, from about 20-80%, from about 25-75%, from about 30-70%, from about 5-10%, from about 6-15%, from about 5-20%, from about 5-25%, from about 5-30%, from about 5-35%, from about 5-40%, from about 50-95%, from about 55-95%, from about 60-95%, from about 65-95%, from about 70-95%, from about 75-95%, from about 80-95%, or from about 85-95%, by weight, based on the weight of the hydrolysate, of organic saponifᵢable materials.

Representative hydrolysates of the oil and/or wax may comprise, for example, about 6%, about 10%, about 15%, about 20%, about 25%, about 30%, about 35%, about 40%, about 45%, about 50%, about 55%, about 60%, about 65%, about 70%, about 75%, about 80%, about 85%, or about 90%, by weight, based on the weight of the hydrolysate, of organic saponifiable materials.

Representative hydrolysates may comprise, for example, a hydrolysate of algae oil, amaranth seed oil, anise seed oil, avocado seed oil, barley oil, briza oil, buck wheat oil, candelilla wax, carnuba wax, cassia occidentalis oil, coffee bean oil, de-oiled lecithin, dog fish oil, esparto wax, a fungal oil, a microorganism oil, guayule (plant material extract), jojoba oil, jurinea oil, lanolin, laurel berry oil, olive oil concentrate (phytosqualene), olive seed oil, orange roughy oil, ouricury wax, quinoa seed oil, rye germ oil, shark liver oil, shea butter, sperm whale oil, sugar cane wax, sunflower wax, tall oil, a tall oil distillate, wheat germ oil, rice bran wax, jojoba ester, sunflower oil, mango kernel oil, mango butter, moringa oil, moringa butter, an oil comprising sucrose bonded to 6, 7, or 8 fatty acids, wherein each fatty acid is individually a saturated fatty acid, an unsaturated fatty acid, or a polyunsaturated fatty acid, any derivative(s) thereof, or any combination(s) thereof.

Representative hydrolysates may comprise a hydrolysate of jojoba oil and/or jojoba ester(s) and/or jojoba ester derivative(s).

Representative hydrolysates may be formed, for example, from an oil and/or wax modified by at least one of: alkoxylation, polymerization, acetylation, oxidation, reduction, concentration, distillation, dilution, hydrogenation, partial hydrogenation, interesterification, double bond modification, randomization, refinement, enzymatic treatment, passive natural processes, biological processes, or any combination(s) thereof

Materials and methods for forming representative hydrolysates in general conformance with the present invention are representatively disclosed in U.S. Pat. No. 7,435,424.

Alcohols

Representative alcohols may be substituted or unsubstituted and may comprise, for example, an alkyl alcohol, a cycloalkyl alcohol, a heterocycloalkyl alcohol, an aryl alcohol, a heteroaryl alcohol, an arylalkyl alcohol, a heteroarylalkyl alcohol, a cycloalkyl alkyl alcohol, a diol, a triol, a bicyclic alcohol, a tricyclic alcohol, a tetracyclic alcohol, a bicyclic alkyl alcohol, a tricyclic alkyl alcohol, a tetracyclic alkyl alcohol, any derivative(s) thereof, or any combination(s) thereof.

Representative alcohols may include, for example, substituted or unsubstituted: methanol, ethanol, iso-propanol, n-propanol, n-butanol, iso-butanol, sec-butanol, tert-butanol, n-pentanol, n-hexanol, phenol, benzyl alcohol, 1-phenyl ethanol, 1-phenyl-1-methyl ethanol, 2-phenyl ethanol, 3-phenyl propanol, 2-phenyl propanol, 1-phenyl propanol, 1-phenyl-2-propanol, cyclooctanol, cycloheptanol, cyclohexanol, cyclopentanol, cyclobutanol, cyclopropanol, cyclooctyl methyl alcohol, cycloheptyl methyl alcohol, cyclohexyl methyl alcohol, cyclopentyl methyl alcohol, cyclobutyl methyl alcohol, cyclopropyl methyl alcohol, 2-cyclooctyl ethanol, 2-cycloheptyl ethanol, 2-cyclohexyl ethanol, 2-cyclopentyl ethanol, 2-cyclobutyl ethanol, 2-cyclopropyl ethanol, 1-cyclooctyl ethanol, 1-cycloheptyl ethanol, 1-cyclohexyl ethanol, 1-cyclopentyl ethanol, 1-cyclobutyl ethanol, 1-cyclopropyl ethanol, 1-cyclooctyl-1-methyl ethanol, 1-cycloheptyl-1-methyl ethanol, 1-cyclohexyl-1-methyl ethanol, 1-cyclopentyl-1-methyl ethanol, 1-cyclobutyl-1-methyl ethanol, 1-cyclopropyl-1-methyl ethanol, 3-cyclooctyl propanol, 3-cycloheptyl propanol, 3-cyclohexyl propanol, 3-cyclopentyl propanol, 3-cyclobutyl propanol, 3-cyclopropyl propanol, 2-cyclooctyl propanol, 2-cycloheptyl propanol, 2-cyclohexyl propanol, 2-cyclopentyl propanol, 2-cyclobutyl propanol, 2-cyclopropyl propanol, 1-cyclooctyl propanol, 1-cycloheptyl propanol, 1-cyclohexyl propanol, 1-cyclopentyl propanol, 1-cyclobutyl propanol, 1-cyclopropyl propanol, 1,2-propane diol, 1,3-propane diol, glycerol, a monoglyceride, a diglyceride, any derivative(s) thereof, or any combination(s) thereof.

Representative alcohols may comprise, for example, substituted or unsubstituted phenol, pyrocatechol, resorcinol, 1,3,5-benzenetriol, 2,6-naphthalenediol, 1-naphthol, 2-naphthol, 1,6-naphthalenediol, 2,7-naphthalenediol, 1,2,3-benzenetriol, 1,2,4-benzenetriol, 1,2,3,4,5,6-benzenehexol, 2-ethyl-6-methylphenol, butylated hydroxytoluene, any derivative(s) thereof, or any combination(s) thereof.

Representative alcohols may include, for example, substituted or unsubstituted cedrenol, cedrol, 1-cyclopentylethanol, 3-methylcyclopentanol, 1-ethylcyclopentanol, 1-methylcyclopentanol, cyclopentane-1,3-diyldimethanol, cyclopentylmethanol, 3-cyclopentyl-1-propanol, 3-methyl-1,2-cyclopentanediol, tricyclo[2.2.1.0(2,6)]heptane-3,5-diol, 5-norbornen-2-ol, endo-5-norbornen-2-ol, exo-5-norbomen-2-ol, 1,2,3,5-tetraphenyl-1,2-cyclopentanediol, 1,2,3-triphenyl-2,3,8,8a-tetrahydrocyclopenta[a]indene-3,3a,8(1H)-triol, 3,3a-dihydroxy-1,2,3-triphenyl-2,3,3a,8a-tetrahydrocyclopenta[a]inden-8(1H)-one, 3,3a-dihydroxy-1-(4-methoxyphenyl)-2,3-diphenyl-2,3,3a,8a-tetrahydrocyclopenta[a]inden-8(1H)-one, 3,3a-dihydroxy-1,2,3-tris(4-methoxyphenyl)-2,3,3a,8a-tetrahydrocyclopenta[a]inden-8(1H)-one, 1,4-cyclohexanediol, 3,3,5,5-tetramethylcyclohexanol, 3,3-dimethylcyclohexanol, 3,3,5-trimethylcyclohexanol, 2-methylcyclohexanol, 2,4-dimethylcyclohexanol, 1,3-cyclohexanediol, 2,5-dimethylcyclohexanol, 3,5-dimethylcyclohexanol, 4-methylcyclohexanol, 3-methylcyclohexanol, 1-cyclohexyl-1-propanol, 3-cyclohexyl-2-butanol, 2-butylcyclohexanol, 1-cyclohexyl-1-butanol, 4-ethylcyclohexanol, 2-(2-ethylbutyl)cyclohexanol, 2-propylcyclohexanol, 2-methyl-1,4-cyclohexanediol, 1-(4-methylcyclohexyl)ethanol, 4-butylcyclohexanol, 2-ethylcyclohexanol, 1-cyclohexylethanol, 2,3-dimethylcyclohexanol, 2-isopropyl-5-methylcyclohexanol, 1-cyclohexyl-3-methyl-1-butanol, 4-propylcyclohexanol, cyclohexane-1,4-dimethanol, cyclohexane-1,3-dimethanol, 1-methylcyclohexanol, cyclohexylmethanol, 4,6-dimethyl-1,3-cyclohexanediol, 4-cyclohexyl-1-butanol, 3-cyclohexyl-1-propanol, 2-cyclohexylethanol, (2,6-dimethylcyclohexyl)methanol, amino(4-hydroxycyclohexyl)acetic acid, exo-norborneol, (+)-endo-2-norborneol, endo-norborneol, alpha-norborneol, borneol, (+)-borneol, (−)-borneol, fenchol, (+)-fenchol, (−)-fenchol, 3-[(1R,4R)-5,5,6-trimethylbicyclo[2.2.1]hept-2-yl]cyclohexanol, tricyclo(5.2.1.02,6)decane-3,4-diol, tricyclo[2.2.1.0(2,6)]heptane-3,5-diol, 8-hydroxytricyclo(5.2.1.02,6)-4-decene, N,N-dicyclohexyl[(1R,2S,4S)-2-hydroxy-7,7-dimethylbicyclo[2.2.1]hept-1-yl]methanesulfonamide, (1R,2S,3R,4S)-1,7,7-trimethyl-3-(4-morpholinyl)bicyclo[2.2.19heptan-2-ol, N-(3,5-dimethylphenyl)-N-[(1S,2R,3S,4R)-3-hydroxy-4,7,7-trimethylbicyclo[2.2.1]hept-2-yl]benzene sulfonamide, bicyclo[2.2.2]octan-2-ol, bicycle[2.2.2]octane-2,3-dimethanol, bicyclo[2.2.2]octane-1,4-diol, 7-hydroxy-3,5,5-trimethylbicyclo[2.2.2]octan-2-one, 7-hydroxy-5,5-dimethylbicyclo[2.2.2] octan-2-one, 1-hydroxy-4-methylbicyclo [2.2.2]octan-2-one, any derivative(s) thereof, or any combination(s) thereof.

Representative alcohols may include, for example,

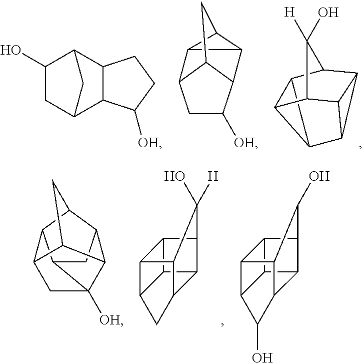

any derivative(s) thereof, or any combination(s) thereof.

Representative alcohols may further comprise, for example, benzyl alcohol, 2-phenyl ethanol, 3-phenyl propanol, cedrenol, cedrol, jojoba alcohol, borneol, iso-borneol, dodecanol, iso-dodecanol, icosanol, iso-icosanol, the alcohol portion of any wax/oil hydrolysate, any derivative(s) thereof, or any combination(s) thereof.

The alcohol may be an alcohol of formula (1):

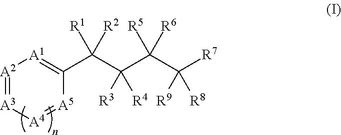

wherein,
n is 0 or 1
A¹, A², A³, A⁴, and A⁵ are each, independently, N, C, S, O, P, or B;
R¹, R², R³, R⁴, R⁵, R⁶, R⁷, R⁸, and R⁹ are each, independently, H, OH, alkyl which can be linear or branched and substituted or unsubstituted and interrupted in its chain by O, S, S(O), and/or S(O)₂, cycloalkyl, cycloalkyl alkyl, aryl, arylalkyl, heteroaryl, heteroaryl alkyl, heterocycloalkyl, or heterocycloalkyl alkyl;
with the provisos that:

at least one of R¹, R², R³, R⁴, R⁵, R⁶, R⁷, R⁸, and R⁹ is an OH group and/or at least one of A¹, A², A³, A⁴ and A⁵ is a carbon substituted with an OH group;
each of A¹, A², A³, A⁴ and A⁵ may, independently, be unsubstituted or substituted with a substituent that is H, OH, alkyl which can be linear or branched and substituted or unsubstituted and interrupted in its chain by O, S, S(O), and/or S(O)₂, cycloalkyl, cycloalkyl alkyl, aryl, arylalkyl, heteroaryl, heteroaryl alkyl, heterocycloalkyl, or heterocycloalkyl alkyl; and
that any R group, together with a different R group, and any intervening carbon atoms, can be combined to form a ring, so long as the 2 R groups that form the ring are both not hydrogen.

Buffer Substances

Representative buffer substances may include, for example, a zwitter ion, an addition salt of an organic molecule, an organic acid, an organic base, an inorganic acid, an inorganic base, an amino acid, an organic polymer, an inorganic polymer, a copolymer, a block copolymer, an inorganic salt, an organic salt, an oligomer comprising two to ten amino acids wherein the amino acids can be the same or different, any derivative(s) thereof, or any combination(s) thereof.

Representative buffer substances may comprise, for example, an acetone oxime, adenosine, an adenosine phosphate, alizarin sulfonic acid, aminobenzoylhydrazide, aminodecylphosphonic acid, ammonia, arginine, ascorbic acid, benzenediazine, benzyl-alpha-dioxime, benzimidazole, benzoyl hydrazine, benzoyl pyruvic acid, besylic acid, biguanidine, boric acid, carbonic acid, chrome azurol S, chrome dark blue, cyanoacetohydrazide, cytidine phosphoric acid, cytosine, dicyclohexyl amine, diethylbiguanide, diguanidine, dihexylamine, dihydroxybenzene disulfonic acid, dihydroxypurine, dimethylbiguanide, dimethylethanethiol, eriochrome black T, esylic acid, ethylbiguanide, ethylenebiguanide, glucoascorbic acid, glutarimide, guanine, hypochlorous acid, histamine, hydroxybenzaldehyde oxime, N-(hydroxyethyl)biguanide, hydroxypyridine, hydroxyquinoline, hypoxanthene, inosine, iodohistamine, maleic acid, malic acid, malonic acid, mercapto histidine, mesylic acid, octopine, oxalic acid, phenylalanyl arginine, phosphoric acid, pyruvic acid, ribose-5-phosphonic acid, salicyclic acid, serine, sulfonic acid, tartaric acid, tosylic acid, triflic acid, tyrosyl, xanthosine, xanthosine monophosphate, zincon, any derivative(s) thereof, or any combination(s) thereof.

Representative buffer substances may include, for example, a phosphinic acid, for example a C₁, C₂, C₃, C₄, C₅, C₆, C₇, C₈, C₉, or C₁₀ alkyl phosphinic acid, sulfuric acid, a salt of phosphoric acid, a salt of sulfuric acid, an ester of phosphoric acid, an ester of sulfuric acid, ammonium sulfate mono basic, ammonium sulfate di basic, lithium sulfate mono basic, lithium sulfate di basic, potassium sulfate mono basic, potassium sulfate di basic, sodium sulfate mono basic, sodium sulfate di basic, calcium sulfate, magnesium sulfate, a carbonate, a mono ester of a carbonate, a di ester of a carbonate, sodium bicarbonate, sodium carbonate, lithium bicarbonate, lithium carbonate, potassium bicarbonate, potassium carbonate, magnesium carbonate, calcium carbonate, mono potassium phosphate, di potassium sulfate, tri potassium phosphate, mono sodium phosphate, di sodium phosphate, tri sodium phosphate, mono lithium phosphate, di lithium phosphate, tri lithium phosphate, calcium phosphate, magnesium phosphate, polyphosphoric acid, N,N-bis(2-hydroxyethyl)-2-aminoethanesulfonic acid, N,N-bis(2-hydroxyethyl)glycine, 4-(2-hydroxyethyl)piperazine-1-ethanesulfonic acid, 2-amino-2-methyl-1-propanol hydrochloride, 2-amino-2-methyl-1-propanol, 2-amino-2-methyl-1,3-propanediol, lactic acid, L-(+)-lactic acid, D-(−)-lactic acid, L-(−)-malic acid, D-(+)-malic acid, L-β-imidazolelactic acid, N-(2-acetamido)-2-aminoethanesulfonic acid, N-(2-acetamido)-2-iminodiacetic acid, N-(2-acetamido)iminodiacetic acid monosodium salt, 3-([1,1-dimethyl-2-hydroxyethyl]amino)-2-hydroxypropanesulfonic acid, ammonium phosphate, ammonium sulfate, ammonium acetate, lithium acetate, sodium acetate, potassium acetate, calcium acetate, magnesium acetate, ammonium chloride, citric acid, ammonium citrate mono basic, ammonium citrate di basic, ammonium citrate tri basic, lithium citrate mono basic, lithium citrate di basic, lithium citrate tri basic, potassium citrate mono basic, potassium citrate di basic, potassium citrate tri basic, calcium citrate, magnesium citrate, an ester of tartaric acid, a salt of tartaric acid, L-(+)-tartaric acid, a salt of L-(+)-tartaric acid, an ester of L-(+)-tartaric acid, D-(−)-tartaric acid, a salt of D-(−)-tartaric acid, an ester of D-(−)-tartaric acid, ammonium tartrate mono basic, ammonium tartrate di basic, lithium tartrate mono basic, lithium tartrate di basic, potassium tartrate mono basic, potassium tartrate di basic, sodium tartrate mono basic, sodium tartrate di basic, calcium tartrate, magnesium tartrate, N,N-bis(2-hydroxyethyl)-2-aminoethanesulfonic acid sodium salt, N,N-bis(2-hydroxyethyl)-2-aminoethanesulfonic acid potassium salt, N,N-bis(2-hydroxyethyl)-2-aminoethanesulfonic acid lithium salt, 2,2-bis(hydroxymethyl)-2,2′,2″-nitrilotriethanol, a salt of 2,2-bis(hydroxymethyl)-2,2′,2″-nitrilotriethanol, an ester of 2,2-bis(hydroxymethyl)-2,2′,2″-nitrilotriethanol, 1,3-bis[tris(hydroxymethyl)methylamino]propane, a salt of 1,3-bis[tris(hydroxymethyl)methylamino]propane, boric acid, a salt of boric acid, an ester of boric acid, 4-(cyclohexylamino)-1-butanesulfonic acid, a salt of 4-(cyclohexylamino)-1-butanesulfonic acid, a sulfonate ester of 4-(cyclohexylamino)-1-butanesulfonic acid, 3-(cyclohexylamino)-2-hydroxy-1-propanesulfonic acid, a salt of 3-(cyclohexylamino)-2-hydroxy-1-propanesulfonic acid, a sulfate ester of 3-(cyclohexylamino)-2-hydroxy-1-propanesulfonic acid, 3-(N,N-bis[2-hydroxyethyl]amino)-2-hydroxypropanesulfonic acid, a salt of 3-(N,N-bis[2-hydroxyethyl]amino)-2-hydroxypropanesulfonic acid, a sulfate ester of 3-(N,N-bis[2-hydroxyethyl]amino)-2-hydroxypropanesulfonic acid, 2,2′-iminodiethanol, isoleucine, leucine, lysine, methionine, phenylalanine, threonine, tryptophan, valine, alanine, asparagine, aspartic acid, cysteine, glutamic acid, glutamine, glycine, proline, serine, tyrosine, arginine, histidine, ornithine, taurine, 3-(cyclohexylamino)-1-propanesulfonic acid, 2-(cyclohexylamino)ethanesulfonic acid, 4-(2-hydroxyethyl)-1-piperazinepropanesulfonic acid, disodium ethylenediaminetetraacetate dihydrate, 2-aminoethanol, 2-aminoethanol hydrochloride, disodium ethylenediaminetetraacetate dihydrate, ethylenedinitrilo)tetraacetic acid, hydroxyacetic acid, N-(2-hydroxyethyl)piperazine-N′-(4-butanesulfonic acid), imidazole, imidazole hydrochloride, 2-(N-morpholino)ethanesulfonic acid hemisodium salt, 2-(N-morpholino)ethanesulfonic acid hydrate, 2-(N-morpholino)ethanesulfonic acid potassium salt, 4-(N-morpholino)butanesulfonic acid, 3-(N-morpholino)propanesulfonic acid hemisodium salt, maleic acid, 1,4-piperazinediethanesulfonic acid dipotassium salt, piperazine-1,4-bis(2-hydroxypropanesulfonic acid) dihydrate, 2-amino-2-(hydroxymethyl)-1,3-propanediol, lactic acid sodium salt, sodium borate decahydrate, N-tris(hydroxymethyl)methyl-4-aminobutanesulfonic acid, N-tris(hydroxymethyl)methyl-3-aminopropanesulfonic acid sodium and/or potassium salt, 3-(N-tris[hydroxymethyl]methylamino)-2-hydroxypropanesulfonic acid, 2-[(2-hydroxy-1,1-bis[hydroxymethyl]ethyl)amino]ethanesulfonic acid, N-[tris(hydroxymethyl)methyl]glycine, meglumine, phthalic acid, potassium hydrogen phthalate, sodium hydrogen phthalate, lithium hydrogen phthalate, magnesium phthalate, calcium phthalate, di potassium phthalate, di lithium phthalate, di sodium phthalate, or any derivative(s) or combination(s) thereof.

Representative buffer substances may be a polymer, an organic polymer, an inorganic polymer, a copolymer, an organic copolymer, a block copolymer, an organic block copolymer, any derivative thereof, or any combination thereof. The polymer, organic polymer, inorganic polymer, copolymer, organic copolymer, block copolymer, organic block copolymer, or any derivative thereof, can have a weight average molecular weight, for example, of from about 50 to about 20,000,000.

Representative weight average molecular weight(s) can include, for example, about 100, about 200, about 300, about 400, about 500, about 600, about 700, about 800, about 900, about 1,000, about 2,000, about 3,000, about 4,000, about 5,000, about 6,000, about 7,000, about 8,000, about 9,000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, about 1,000,000, about 2,000,000, about 3,000,000, about 4,000,000, about 5,000,000, about 6,000,000, about 7,000,000, about 8,000,000, about 9,000,000, about 10,000,000, about 11,000,000, about 12,000,000, about 13,000,000, about 14,000,000, about 15,000,000, about 16,000,000, about 17,000,000, about 18,000,000, about 19,000,000 or about 20,000,000.

Representative weight average molecular weight(s) can range, for example, from about 1,000 to about 20,000,000, from about 10,000 to about 20,000,000, from about 50,000 to about 20,000,000, from about 100,000 to about 20,000,000, from about 250,000 to about 20,000,000, from about 500,000 to about 20,000,000, from about 1,000,000 to about 20,000,000, from about 5,000,000 to about 20,000,000, from about 10,000,000 to about 20,000,000, or from about 15,000,000 to about 20,000,000.

Representative weight average molecular weight(s) can be, for example, at least 100, at least 200, at least 300, at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 20,000, at least 30,000, at least 40,000, at least 50,000, at least 60,000, at least 70,000, at least 80,000, at least 90,000, at least 100,000, at least 200,000, at least 300,000, at least 400,000, at least 500,000, at least 600,000, at least 700,000, at least 800,000, at least 900,000, at least 1,000,000, at least 2,000,000, at least 3,000,000, at least 4,000,000, at least 5,000,000, at least 6,000,000, at least 7,000,000, at least 8,000,000, at least 9,000,000, at least 10,000,000, at least 11,000,000, at least 12,000,000, at least 13,000,000, at least 14,000,000, at least 15,000,000, at least 16,000,000, at least 17,000,000, at least 18,000,000, at least 19,000,000 or at least 20,000,000.

Representative buffer substances may comprise a polymer, which can be, for example and without limitation, a polyethylene imine (PEI) of various molecular weights, a polyvinyl imidazole (PVI) of various molecular weights, and/or the like. The polyethylene imine can be a linear polyethylene imine and/or a branched polyethylene imine, or any derivative(s) thereof. The branched polyethylene imine can contain primary, secondary and/or tertiary amino groups. The polyethylene imine can be synthesized, for example, by polymerization of azeridine and/or by hydrolysis of synthesis of poly(2-ethyl-2-oxazoline). The amino groups on the polyethylene imine can be wholly or partially protonated.

Representative buffer substances may include, for example, a block copolymer comprising blocks of linear polyethylene imine and branched polyethylene imine.

Representative buffer substances can be an amino acid polymer or any derivative(s) thereof The buffer substance can be, for example, polylysine, polyarginine, polyhistadine, polyaspartic acid, polyglutamic acid, polyornithine, any derivative thereof, or any combination thereof. The amino groups on polylysine, polyargine, polyhistadine, and/or polyornithine can be wholly or partially protonated. The carboxyl groups on polyaspartic acid and/or polyglutamic acid can be wholly or partially deprotonated (and hence anionic) with, for example, sodium, lithium, ammonium and/or potassium counter ions.

Representative buffer substances may comprise an acrylate polymer or any derivative(s) thereof The acid groups on the polymer can be wholly or partially deprotonated. The acrylate polymer can be, for example, a polymer of acrylic acid, methacrylic acid, or ethacrylic acid, or derivative(s) thereof. The acid groups in the polymer can be all or partially anionic with, for example, sodium, lithium, ammonium and/or potassium counter ions.

Representative buffer substances may include an oligomer, containing, for example, from 2 to 10 amino acids or derivative(s) thereof. The oligomer can contain, for example, 3 amino acids, 4 amino acids, 5 amino acids, 6 amino acids, 7 amino acids, 8 amino acids, or 9 amino acids, or any derivatives thereof. The oligomer can be an oligomer of glycine (gly), for example gly-gly, gly-gly-gly, gly-gly-gly-gly, and/or gly-gly-gly-gly-gly, any derivative(s) thereof, or any combination(s) thereof.

Representative buffer substances can include a copolymer of at least two of acrylic acid, methacrylic acid, and/or ethacrylic acid. The acid groups in the copolymer can be all or partially anionic with, for example, sodium, lithium, ammonium and/or potassium counter ions.

Representative buffer substances can comprise a block copolymer, wherein each block of the block copolymer can comprise acrylic acid, methacrylic acid, or ethacrylic acid, so long as the block copolymer contains at least two non-identical blocks.

Representative buffer substances may be in the form of a hydrate, for example, a mono hydrate, a di hydrate, a tri hydrate, a tetra hydrate, a penta hydrate, a hexa hydrate, a hepta hydrate, an octa hydrate, a nona hydrate, or a deca hydrate.

Hydroxide Salts

The hydroxide salt can be, for example, any inorganic and/or organic hydroxide salt.

The hydroxide salt can be, for example, lithium hydroxide, potassium hydroxide, sodium hydroxide, magnesium hydroxide, calcium hydroxide, zinc (II) hydroxide, iron (II) hydroxide, iron (III) hydroxide, manganese hydroxide, ammonium hydroxide, aluminum hydroxide, alumina hydroxide, barium hydroxide, benzethonium hydroxide, benzyltrimethylammoniurn hydroxide, cerium (IV) hydroxide, cesium hydroxide, choline hydroxide, cobalt (II) hydroxide, copper (II) hydroxide, hydroxyl amine, diethyldimethylammonium hydroxide, dimethyldodecylethylammonium hydroxide, hexadecyltrimethylammonium hydroxide, hexamethonium hydroxide, methyltripropylammonium hydroxide, tetrabutylammonium hydroxide, tetrabutylphosphonium hydroxide, tetraethylammonium hydroxide, tetrahexylammonium hydroxide, tetramethylammonium hydroxide, tetraoctadecylammonium hydroxide, tetraoctylammonium hydroxide, tetrapentylammonium hydroxide, tetrapropylammonium hydroxide, tributylmethylammonium hydroxide, triethylmethylammonium hydroxide, trihexyltetradecylammonium hydroxide, trimethylphenylammonium hydroxide, trimethylsulfonium hydroxide, zirconium (IV) hydroxide, 1-ethyl-3-methylimidazolium hydroxide, any derivative(s) thereof, and any combination(s) or valence state variant(s) thereof.

Water

Sources of water are not limited and may be, for example, tap water, purified water, distilled water, de-ionized water, spring water, mineral water, reverse osmosis purified water, water purified by chlorination, boiled water, soft water, hard water, rain water, rose water, sea water, salt water, water from melting and/or melted snow, water from melting and/or melted ice, water formed in situ in a chemical reaction, and/or any combination(s) thereof

Optionally Excluded Ingredients

A thiol, a thiol containing molecule or moiety, a salt of a thiol and/or a salt of a thiol-containing molecule may optionally be excluded from the composition.

A disulfide containing molecule or moiety and/or a salt thereof may be optionally excluded from the composition.

An aldehyde containing molecule or moiety, such as formaldehyde, and/or a salt thereof, may be optionally excluded from the composition.

A formalehyde donor containing molecule or moiety, and/or a salt thereof, may be optionally excluded from the composition.

Ammonia and/or an ammonia-containing molecule or moiety, including salts thereof, may be optionally excluded from the composition.

Ingredient Amounts & Ratios

Compositions in accordance with various representative embodiments of the present invention may contain an alcohol such that a ratio of alcohol to a blend of water, buffer substance, hydrolysate and hydroxide can range, for example, from about 1:1 to about 1:100 on a weight to weight basis. For example, the ratio of the alcohol to the blend can be about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:51, about 1:52, about 1:53, about 1:54, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, or about 1:99, on a weight-to-weight basis.

The ratio of the alcohol to the blend of water, buffer substance, hydrolysate and hydroxide can range, for example, from about 1:1 to about 1:90, from about 1:1 to about 1:80, from about 1:1 to about 1:70, from about 1:1 to about 1:60, from about 1:1 to about 1:50, from about 1:1 to about 1:40, from about 1:1 to about 1:30, from about 1:1 to about 1:20, from about 1:20 to about 1:90, from about 1:30 to about 1:90, from about 1:40 to about 1:90, from about 1:50 to about 1:90, from about 1:60 to about 1:90, from about 1:70 to about 1:90, or from about 1:80 to about 1:90, on a weight-to-weight basis.

Representative compositions in accordance with various embodiments of the present invention may comprise, for example, from about 1% to about 90%, by weight, based on the weight of the composition, of the alcohol. The composition can comprise, for example, from about 5-90%, from about 10-90%, from about 20-90%, from about 25-50%, from about 75-90%, or from about 85-90% of the alcohol, by weight, based on the weight of the composition.

Various representative embodiments of the invention may comprise, for example, about 1%, about 2%, about 3%, about 4%, about 5%, about 6%, about 7%, about 8%, about 9%, about 10%, about 12%, about 14%, about 16%, about 18%, about 20%, about 23%, about 26%, about 29%, about 30%, about 34%, about 38%, about 40%, about 45%, about 50%, about 55%, about 60%, about 65%, about 70%, about 75%, about 80%, about 85%, about 90% of the alcohol, by weight, based on the weight of the composition.

Various representative compositions in accordance with the present invention may comprise from about 1% to about 25% of the alcohol, by weight, based on the weight of the composition. The alcohol may comprise a weight fraction of the composition, for example, corresponding to an amount ranging from about 1% to about 20%, from about 1% to about 15%, from about 1% to about 10%, from about 1% to about 5%, from about 5-25%, from about 10-25%, from about 15-25%, or from about 20-25%, by weight, based on the weight of the composition.

The alcohol may comprise a weight fraction of the composition, for example, corresponding to an amount ranging from about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, by weight, based on the weight of the composition.

Representative embodiments of the present invention may comprise a blend of buffer substance, hydrolysate, water and hydroxide. The blend may comprise a weight fraction of the composition, for example, corresponding to an amount ranging from about 10% to about 99% by weight, based on the weight of the composition. The blend may comprise a weight fraction of the composition, for example, corresponding to an amount ranging from about 15-90%, from about 20-85%, from about 25-80%, from about 20-75%, from about 15-70%, from about 20-65%, from about 25-60%, from about 30-55%, from about 35-50%, from about 40-45%, from about 10-99%, from about 15-99%, from about 20-99%, from about 25-99%, from about 30-99%, from about 35-99%, from about 40-99%, or from about 45-99%, by weight, based on the weight of the composition.

Representative blends may optionally comprise water in an amount ranging, for example, from about 0% (e.g., anhydrous) to about 75%, by weight, based on the weight of the blend. The water in the blend may comprise a weight fraction of the composition, for example, corresponding to an amount ranging from about 1-70%, from about 5-65%, from about 10-60%, from about 15-65%, from about 20-60%, from about 25-50%, from about 20-29%, from about 22-29%, from about 24-29%, or from about 26-29%, by weight, based on the weight of the blend.

Representative blends in accordance with various embodiments of the present invention may comprise water in an amount, for example, of about 18%, about 19%, about 20%, about 21%, about 22%, about 23%, about 24%, about 25%, about 26%, about 27%, about 28%, or about 29%, by weight, based on the weight of the blend.

Representative blends can comprise buffer substance in an amount ranging, for example, from 0.5-25%, by weight, based on the weight of the blend. The buffer substance can be comprised in the blend, for example, in an amount ranging from about 0.5-1%, from about 0.5-2%, from about 0.5-3%, from about 0.5-4%, from about 0.5-5%, from about 0.5-6%, from about 0.5-7%, from about 0.5-8%, from about 0.5-9%, from about 0.5-10%, from about 0.5-11%, from about 0.5-12%, from about 0.5-13%, from about 0.5-14%, from about 0.5-15%, from about 0.5-16%, from about 0.5-17%, from about 0.5-18%, from about 0.5-19%, from about 0.5-20%, from about 0.5-21%, from about 0.5-22%, from about 0.5-23%, from about 0.5-24%, from about 1-25%, from about 2-25%, from about 3-25%, from about 4-25%, from about 5-25%, from about 6-25%, from about 7-25%, from about 8-25%, from about 9-25%, from about 10-25%, from about 11-25%, from about 12-25%, from about 13-25%, from about 14-25%, from about 15-25%, from about 16-25%, from about 17-25%, from about 18-25%, from about 19-25%, from about 20-25%, from about 21-25%, from about 22-25%, from about 23-25%, or from about 24-25%, by weight, based on the weight of the blend.

The blend may comprise buffer substance in an amount, for example, of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, by weight, based on the weight of the blend.

The blend may comprise hydrolysates (i.e., organic unsaponifiable materials in combination with organic saponifiable materials) in an amount, for example, from about 3-40%, by weight, based on the weight of the blend. The blend may comprise hydrolysates in an amount, for example, ranging from about 5-40%, from about 10-40%, from about 15-40%, from about 20-40%, from about 25-40%, from about 30-40%, from about 35-40%, from about 5-35%, from about 5-30%, from about 5-25%, from about 5-20%, from about 5-15%, from about 5-10%, from about 10-35%, from about 15-30%, or from about 20-25%, by weight, based on the weight of the blend.

Representative embodiments of the blend can comprise hydrolysates in an amount, for example, of about 3%, about 5%, about 7%, about 9%, about 11%, about 13%, about 15%, about 17%, about 19%, about 21%, about 23%, about 25%, about 27%, about 29%, about 31%, about 33%, about 35%, about 37%, or about 39%, by weight, based on the weight of the blend.

The blend may further comprise hydroxide salts in an amount, for example, ranging from about 18-29%, by weight, based on the weight of the blend. For example, hydroxide salts may comprise an amount ranging from about 20-27%, from about 22-25%, from about 18-27%, from about 18-25%, from about 18-23%, from about 18-20%, from about 20-29%, from about 22-29%, from about 25-29%, or from about 27-29%, by weight, based on the weight of the blend.

The blend may comprise hydroxide salts in an amount, for example, of about 18%, about 19%, about 20%, about 21%, about 22%, about 23%, about 24%, about 25%, about 26%, about 27%, about 28%, or about 29%, by weight, based on the weight of the blend.

In a representative embodiment, the composition comprises benzyl alcohol as the alcohol, potassium hydroxide as the hydroxide salt, tri potassium phosphate as the buffer substance, and jojoba oil hydrolysate as the hydrolysate.

In another representative embodiment, the composition comprises cedrol as the alcohol, potassium hydroxide as the hydroxide salt, tri potassium phosphate mono hydrate as the buffer substance, and the hydrolysis products of jojoba oil (i.e., jojoba oil hydrolysate) as the hydrolysate.

In yet another representative embodiment, the composition comprises 2-phenyl ethanol as the alcohol, sodium hydroxide as the hydroxide salt, tri potassium phosphate as the buffer substance, and jojoba oil hydrolysate as the hydrolysate.

In a further representative embodiment, the composition comprises 3-phenyl propanol as the alcohol, potassium hydroxide as the hydroxide salt, tri potassium phosphate as the buffer substance, and spermaceti hydrolysate as the hydrolysate.

In another representative embodiment, the composition comprises cedrenol as the alcohol, potassium hydroxide as the hydroxide salt, tri potassium phosphate as the buffer substance, and jojoba oil hydrolysate as the hydrolysate.

In still another representative embodiment, the composition comprises benzyl alcohol as the alcohol, potassium hydroxide as the hydroxide salt, a polyethylene imine (PEI) as the buffer substance, and jojoba oil hydrolysate as the hydrolysate.

In yet another representative embodiment, the composition comprises 2-phenyl ethanol as the alcohol, potassium hydroxide as the hydroxide salt, a polyethylene imine (PEI) as the buffer substance, and jojoba oil hydrolysate as the hydrolysate.

In still yet another representative embodiment, the composition comprises 3-phenyl propanol as the alcohol, potassium hydroxide as the hydroxide salt, a polyethylene imine (PEI) as the buffer substance, and jojoba oil hydrolysate as the hydrolysate.

In another representative embodiment, the composition comprises cedrol as the alcohol, potassium hydroxide as the hydroxide salt, a polyethylene imine (PEI) as the buffer substance, and hydrolyzed jojoba esters (i.e., jojoba oil hydrolysate) as the hydrolysate.

In some representative embodiments, the composition may further comprise an additional ingredient, which can be, for example, an excipient. The composition can comprise, for example, from about 1-90% by weight of the additional ingredient, which can be an excipient, and from about 10-99% by weight of a combination of the blend and the alcohol.

In some embodiments, the composition can comprise, for example, about 99% by weight, about 98% by weight, about 97% by weight, about 96% by weight, about 95% by weight, about 94% by weight, about 93% by weight, about 92% by weight, about 91% by weight, about 90% by weight, about 85% by weight, about 80% by weight, about 75% by weight, about 70% by weight, about 65% by weight, about 60% by weight, about 55% by weight, about 50% by weight, about 45% by weight, about 40% by weight, about 35% by weight, about 30% by weight, about 25% by weight, about 20% by weight, about 15% by weight, or about 10% by weight of a combination of the blend and the alcohol, where the % by weight is based on the total weight of the composition.

In some embodiments, the composition can comprise, for example, about 10-98% by weight, about 10-97% by weight, about 10-96% by weight, about 10-95% by weight, about 10-94% by weight, about 10-93% by weight, about 10-92% by weight, about 10-91% by weight, about 10-90% by weight, about 10-80% by weight, about 10-70% by weight, about 10-60% by weight, about 10-50% by weight, about 10-40% by weight, about 10-30% by weight, or about 10-20% by weight of the blend and the alcohol, where the % by weight is based on the total weight of the composition.

In some embodiments, the composition can comprise, for example, about 20-99% by weight, about 30-99% by weight, about 40-99% by weight, about 50-99% by weight, about 60-99% by weight, about 70-99% by weight, about 80-99% by weight, about 90-99% by weight, about 91-99% by weight, about 92-99% by weight, about 93-99% by weight, about 94-99% by weight, about 95-99% by weight, about 96-99% by weight, or about 97-99% by weight of the blend and the alcohol, where % by weight is based on the total weight of the composition.

For example, the composition can comprise an additional ingredient, which can be an excipient, in an amount of about 1% by weight, about 2% by weight, about 3% by weight, about 4% by weight, about 5% by weight, about 6% by weight, about 7% by weight, about 8% by weight, about 9% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, or about 85% by weight, based on the weight of the composition, of the additional ingredient, which can be, for example, an excipient.

The composition can comprise the additional ingredient, which can be an excipient, for example, in an amount ranging from about 2-90% by weight, about 3-90% by weight, about 4-90% by weight, about 5-90% by weight, about 6-90% by weight, about 7-90% by weight, about 8-90% by weight, about 9-90% by weight, about 10-90% by weight, about 20-90% by weight, about 30-90% by weight, about 40-90% by weight, about 50-90% by weight, about 60-90% by weight, about 70-90% by weight, or about 80-90% by weight, based on the weight of the composition.

The composition can comprise the additional ingredient, which can be an excipient, for example, in an amount ranging from about 1-80% by weight, about 1-70% by weight, about 1-60% by weight, about 1-50% by weight, about 1-40% by weight, about 1-30% by weight, about 1-20% by weight, about 1-10% by weight, about 1-9% by weight, about 1-8% by weight, about 1-7% by weight, about 1-6% by weight, about 1-5% by weight, about 1-4% by weight, or about 1%-3% by weight, based on the weight of the composition.

The additional ingredient, which can be for example an excipient, may be, for example, a viscosity modifier, a stability enhancer, an emulsifier, an aesthetic feel enhancer, an anti-oxidant, a glidant, a lubricant, an anti-microbial, an anti-fungal, a preservative, a diluent, a filler, a solubility enhancer, or any combination thereof

The additional ingredient, which can be for example an excipient, may be, for example, an aluminum oxide, an aluminum stearate, ammonium alginate, bentonite, attapulgite, a benzalkonium chloride, a benzethonium chloride, calcium alginate, calcium stearate, carboxymethylcellulose calcium, carboxymethylcellulose sodium, microcrystalline cellulose, cellulose powdered, cellulose acetate, cellulose acetate phthalate, cetrimide, cetylpyridinium chloride, chitosan, colloidal silicon dioxide, croscarmellose sodium, denatonium benzoate, a dextrate, a dextrin, a dextrose, gelatin, guar gum, hectorite, hexetidine, hypromellose, hypromellose acetate succinate, hypromellose phthalate, inulin, an iron oxide, a lactose, magnesium 15 hydroxystearate, magnesium aluminum silicate, magnesium carbonate, magnesium oxide, magnesium silicate, magnesium stearate, magnesium trisilicate, maltodextrin, mineral oil, polyethylene oxide, a PEGylated molecule, a PEGylated stearate, a PEG-100 stearate, PEG-120 ester of a jojoba wax, potassium metabisulfite, sodium chloride, potassium chloride, magnesium chloride, lithium chloride, calcium chloride, sodium metabisulfite, a starch, zinc stearate, or any combination thereof.

PEG can refer to, for example, polyethylene glycol and/or polypropylene glycol. The PEG in a PEGylated molecule can have a weight average molecular weight of, for example, about 100, about 200, about 300, about 400, about 500, about 600, about 700, about 800, about 900, about 1,000, about 5,000, about 10,000, about 20,000, about 30,000, about 40,000, about 50,000, about 60,000, about 70,000, about 80,000, about 90,000, about 100,000, or about 200,000.

The PEG can have a weight average molecular weight ranging, for example, from about 100-200,000, about 200-200,000, about 300-200,000, about 400-200,000, about 500-200,000, about 600-200,000, about 700-200,000, about 800-200,000, about 900-200,000, about 1,000-200,000, about 5,000-200,000, about 10,000-200,000, about 20,000-200,000, about 30,000-200,000, about 40,000-200,000, about 50,000-200,000, about 60,000-200,000, about 70,000-200,000, about 80,000-200,000, about 90,000-200,000, or about 100,000-200,000.

The PEG can have a weight average molecular weight ranging, for example, from about 100-100,000, about 100-90,000, about 100-80,000, about 100-70,000, about 100-60,000, about 100-50,000, about 100-40,000, about 100-30,000, about 100-20,000, about 100-10,000, about 100-5,000, about 100-1,000, about 100-900, about 100-800, about 100-700, about 100-600, about 100-500, about 100-400, about 100-300, or about 100-200.

The PEG in the PEGylated molecule can, for example be covalently attached to the PEGylated molecule. The mode of covalent attachment, for example, without limitation, may comprise an ester, an ether, or an amide bond attachment. The PEG in PEGylated molecule can be, for example, attached to the molecule through an ester bond where an alcohol functional group on the PEG, for example a terminal alcohol functional group, forms an ester bond with a carboxylic acid group on the molecule to which the PEG is attached.

The molecule to which PEG is attached can be, for example, an organic molecule. The molecule can be, for example, and a fatty acid molecule which can, optionally be saturated, optionally be unsaturated, optionally, bear one, or two, or three, or more unsaturated sites, and which can optionally be substituted. The fatty acid molecule can be linear or branched. The fatty acid molecule can contain, for example, from about 1-30 carbon atoms, for example, 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16, carbon atoms, 17 carbon atoms, 18 carbon atoms, 19 carbon atoms, 20 carbon atoms, 21 carbon atoms, 22 carbon atoms, 23 carbon atoms, 24 carbon atoms, 25 carbon atoms, 26 carbon atoms, 27 carbon atoms, 28 carbon atoms, 29 carbon atoms, or 30 carbons atoms.

The composition can be made, for example, by adding, at any point and in any order, an additional ingredient, for example an excipient, in addition to other composition ingredients.

The invention contemplates compositions made by adding an additional ingredient, for example an excipient, to composition ingredients in accordance with various exemplary embodiments of the present invention, for example, as representatively disclosed in Table 1.

Methods of Making Compositions

In a representative embodiment, a method for making compositions in accordance with the present invention may comprise, for example, combining, in any order, water, alcohol, buffer substance, and oil and/or wax hydrolysates to form a mixture, and adjusting the pH of the mixture to give a pH range of about 10-14 when measured in a water solution of 10% by weight of the composition and 90% by weight of water. The pH may be adjusted, for example, by addition of a hydroxide salt, which can be an organic hydroxide salt and/or an inorganic hydroxide salt, to the mixture. The hydroxide salt may be dissolved and/or dispersed and/or suspended in water.

The amounts of water, hydrolysate, alcohol, buffer substance and hydroxide salt employed to make the compositions may be, inter alia for example, any of the amounts or ranges described herein. The hydrolysate employed to make the composition can be, inter alia for example, any hydrolysate provided herein. The amount of organic unsaponifiable materials and organic saponifiable materials in the hydrolysate employed to make the compositions can be any amount or range provided herein. The hydroxide can be, inter alia for example, any hydroxide provided herein. The alcohol can be, inter alia for example, any alcohol provided herein. The buffer substance can be, inter alia for example, any buffer substance provided herein. The water may be, inter alia for example, any form or source of water provided herein.

The temperature at which the compositions are made can range, inter alia for example, from about 0° C. to about 70° C. The temperature can be, inter alia for example, about 5° C., about 10° C., about 15° C., about 20° C., about 25° C., about 30° C., about 40° C., about 45° C., about 50° C., about 55° C., about 60° C., about 65° C., about 70° C., or can be any combination of these temperatures at different stages or steps of manufacture.

The ingredients in the composition can be combined, inter alia for example, by stirring, shaking, agitating, sonicating, or any combination thereof, or any other method of mixing whether now known or hereafter devised in the art.

In some embodiments, ingredients may be combined prior to addition. For example, water can be combined with the hydroxide salt prior to being used to form the compositions, and the resultant water-hydroxide salt solution may be combined, in any order, inter alia for example, with the hydrolysate, the buffer substance, and/or alcohol.

Compositions Made by Methods

Various representative embodiments of compositions in accordance with the present invention may be made by any of the methods provided in this application.

Methods of Relaxing Hair

In a representative embodiment, the disclosed and/or claimed compositions may be employed in a method to relax hair. The amount of curl in hair treated with compositions disclosed herein, relative to control hair that has not been so treated, can decrease, for example, by an amount ranging from about 1-100%.

The percent decrease in the amount of curl in the treated hair, relative to control hair, can be for example, from about 5-100%, from about 10-100%, from about 15-100%, from about 20-100%, from about 25-100%, from about 30-100%, from about 35-100%, from about 40-100%, from about 45-100%, from about 50-100%, from about 55-100%, from about 60-100%, from about 65-100%, from about 70-100%, from about 75-100%, from about 80-100%, from about 85-100%, from about 90-100%, or from about 95-100%.

The percent decrease in the amount of curl in the treated hair, can be, for example, from about 5-95%, from about 5-80%, from about 5-70%, from about 5-60%, from about 5-50%, from about 5-40%, from about 5-30%, from about 5-20%, or from about 5-10%.

In some representative embodiments, hair may be relaxed by applying to the hair any composition disclosed herein. The composition can be applied to the hair, for example, for a time period ranging from about 30 seconds to about 2 hours.

The composition can be applied, for example for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 1 hour, about 1.5 hours, or about 2 hours.

The composition can be applied to the hair, for a time period ranging from about 30 seconds to about 2 minutes, from about 30 seconds to about 3 minutes, from about 30 seconds to about 4 minutes, from about 30 seconds to about 5 minutes, from about 30 seconds to about 6 minutes, from about 30 seconds to about 7 minutes, from about 30 seconds to about 8 minutes, from about 30 seconds to about 9 minutes, from about 30 seconds to about 10 minutes, from about 30 seconds to about 11 minutes, from about 30 seconds to about 12 minutes, from about 30 seconds to about 13 minutes, from about 30 seconds to about 14 minutes, from about 30 seconds to about 15 minutes, from about 30 seconds to about 16 minutes, from about 30 seconds to about 17 minutes, from about 30 seconds to about 18 minutes, from about 30 seconds to about 19 minutes, from about 30 seconds to about 20 minutes, from about 30 seconds to about 21 minutes, from about 30 seconds to about 22 minutes, from about 30 seconds to about 23 minutes, from about 30 seconds to about 24 minutes, from about 30 seconds to about 25 minutes, from about 30 seconds to about 26 minutes, from about 30 seconds to about 27 minutes, from about 30 seconds to about 28 minutes, from about 30 seconds to about 29 minutes, from about 30 seconds to about 30 minutes, from about 30 seconds to about 31 minutes, from about 30 seconds to about 32 minutes, from about 30 seconds to about 33 minutes, from about 30 seconds to about 34 minutes, from about 30 seconds to about 35 minutes, from about 30 seconds to about 36 minutes, from about 30 seconds to about 37 minutes, from about 30 seconds to about 38 minutes, from about 30 seconds to about 39 minutes, from about 30 seconds to about 40 minutes, from about 30 seconds to about 41 minutes, from about 30 seconds to about 42 minutes, from about 30 seconds to about 43 minutes, from about 30 seconds to about 44 minutes, from about 30 seconds to about 45 minutes, from about 30 seconds to about 46 minutes, from about 30 seconds to about 47 minutes, from about 30 seconds to about 48 minutes, from about 30 seconds to about 49 minutes, from about 30 seconds to about 50 minutes, from about 30 seconds to about 55 minutes, from about 30 seconds to about 60 minutes, from about 10 minutes to about 40 minutes, from about 10 minutes to about 35 minutes, from about 10 minutes to about 30 minutes, from about 15 minutes to about 30 minutes, from about 15 minutes to about 35 minutes, or from about 25 minutes to about 40 minutes.

The composition can be applied, for example, using standard application methods known in the art. For example, the composition may be applied by rolling, curling, brushing, smearing, massaging, dipping, spraying, streaking, dropping, sponging, combing, back-combing, painting, via an applicator, or by any combination thereof. In a representative embodiment, the composition may be applied via a syringe. In another representative embodiment, the composition may be manually massaged into the hair. The composition can be massaged into the hair by hand, and the hand can be encased in a glove, for example a plastic, rubber, latex or nitrile glove.

The temperature of the composition when applied to the hair can range, inter alia for example, from about 10-80° C. The temperature of the composition when applied to the hair can range, for example, from about 20° C. to about 70° C., from about 30° C. to about 60° C., or from about 40° C. to about 50° C. The temperature of the composition when applied to hair can be, for example, about 10° C., about 15° C., about 20° C., about 25° C., about 30° C., about 35° C., about 40° C., about 45° C., about 50° C., about 55° C., about 60° C., about 65° C., about 70° C., or about 75° C.

The amount of composition applied to the hair can range, inter alia for example, from about 1 mg -1000 g. The amount can be, for example, about 1 mg, about 10 mg, about 100 mg, about 1 g, about 5 g, about 10 g, about 20 g, about 30 g, about 40 g, about 50 g, about 60 g, about 70 g, about 80 g, about 90 g, about 100 g, about 200 g, about 300 g, about 400 g, about 500 g, about 600 g, about 700 g, about 800 g, about 900 g, or about 1000 g.

The amount of the composition applied to the hair can range, inter alia for example, from about 5 mg, to about 900 g, from about 5 mg to about 800 g, from about 5 mg to about 700 g, from about 5 mg to about 600 g, from about 5 mg to about 500 g, from about 5 mg to about 400 g, from about 5 mg to about 300 g, from about 5 mg to about 200 g, from about 5 mg to about 100 g, from about 5 mg to about 50 g, from about 5 mg to about 25 g, from about 5 mg to about 10 g, or from about 5 mg to about 1 g.

### Hair

Hair to which compositions in accordance with various representative embodiments of the invention is applied can be its natural color or can be colored by a hair colorant. The hair can be, for example, blond, brown, brunette, black, grey, white, blue, red, orange, yellow, green, indigo, violet, pink, purple, or any combination(s) thereof. Hair to which compositions are applied can, but need not, be washed and/or conditioned before the compositions are applied. Hair to which compositions are applied can, but need not, be previously straightened before the compositions are applied. Hair to which compositions are applied can, but need not, be previously chemically treated before the compositions are applied.

The hair can be, for example, human hair, a hair or group of hairs in need of straightening, male or female hair, adult or child hair, attached to an animal, for example a human or other mammal, or can stand alone, for example, being comprised in a wig, a hairpiece, a hair extension, a toupee, or a hair tress. The composition can be applied on all or part of the hair.

The hair can range in length, for example, from about 0.1 inch to about 6 feet. The hair can be of a length, for example, of about 1/10 inch, about ⅕ inch, about ¼ inch, about ⅓ inch, about ½ inch, about 1 inch, about 2 inches, about 3 inches, about 4 inches, about 5 inches, about 6 inches, about 7 inches, about 8 inches, about 9 inches, about 10 inches, about 11 inches, about 12 inches, about 1.5 feet, about 2 feet, about 2.5 feet, about 3.0 feet, about 3.5 feet, about 4.0 feet, about 4.5 feet, about 5.0 feet, or about 5.5 feet.

The hair can correspond to any discrete racial profile or mixed racial profile from a human source. The hair can be, inter alia for example, Caucasian, African, American Indian (including North and South American), African-American, Indian, Latino, Hispanic, Asian, Chinese, Vietnamese, Japanese, Russian, Australian, Aboriginal, North American, South American, European, Afro-Caribbean (found in the Caribbean), Caribbean, Albanian, Arabic, Middle Eastern, Armenian, French, German, Spanish, Portuguese, Basque, Bengali, Berber, Bosniaks, Canadian, Cantonese, Cornish, Croat, Czech, English, Danish, Dogon, Dutch, Finnish, French, Austrian, Greek, Han, Taiwanese, Italian, Igbo, Irish, Jewish, Israeli, Kashmiri, Pakistani, Kazakh, Magyar, Hungarian, Romanian, Thai, Brunei, Singaporian, Malaysian, Indonesian, Mande, Mongol, Mossi, Norwegian, Scandinavian, Punjabi, Polish, Roma, Sarni, Scottish, Serbian, Croatian, Bosnian, Herzegovinian, Slovakian, Slovenian, Swedish, Scandinavian, Tamil, Tartar, Turkish, Welsh, Puerto Rican, or any combination(s) thereof

After the composition is applied to the hair, the hair can be left open to the ambient environment, or can be fully or partially enclosed in a container such as a plastic hair bag optionally containing holes to vent to the atmosphere.

After the composition is applied to the hair, the hair can be mechanically manipulated and/or foiled.

After the composition is applied to the hair for a period of time, some or all of the composition may be removed by rinsing with water and/or a shampoo. Rinsed and/or shampooed hair can be subsequently conditioned and/or colored and/or styled.

The temperature of water employed in rinsing the hair can range, for example, from about 10-50° C. The water temperature can be, for example, about 10° C., about 15° C., about 20° C., about 25° C., about 30° C., about 35° C., about 40° C., about 45° C., or about 50° C.

The shampoo can be, for example, a neutralizing shampoo. A neutralizing shampoo generally operates to ensure the hair straightening effect of the applied relaxer/straightening composition is stopped by quenching the relaxer composition. A neutralizing shampoo can have a pH, for example, of about 1, about 2, about 3, about 4, about 5, about 6, about 7, about 7.4, about 7.5, or about 7.6.

This application contemplates, as inventive embodiments, hair straightened by the methods and/or compositions disclosed herein. The hair may be comprised, for example, in a hair piece, a toupee, a wig, a tress and /or a hair extension.

### Kits

The compositions disclosed herein can be included in a kit. The kit can further comprise a shampoo, a neutralizing shampoo, a neutralizing gel, and/or a conditioner. The kit can contain instructions for physically applying the compositions to hair. The instructions can also include time periods the compositions are to be left applied to hair (i.e., dwell time) and can also include methods for removing the compositions from hair. The instructions may be in any language or in at least two languages. For example, without limitation, the instructions can be in English, Spanish, German, French, Japanese, Russian, Chinese, Portuguese, Vietnamese, or any combination thereof.

The following examples are illustrative and do not limit the scope of the disclosure of the specification or claims.

### EXAMPLES

Representative hair relaxation formulations may be prepared in accordance with Table 2 and Table 3 vide infra. The “FC3” components recited therein correspond to hair relaxer/straighter compositions in accordance with a representative embodiment of the present invention. The FC3 composition is generally intended to be included as one of several components in a finished relaxer formula, as shown, for example, in Table 2 and Table 3.

In an exemplary representative embodiment of the present invention, the “FC3” component of the hair relaxer formulations shown in Table 2 and Table 3 may be manufactured in accordance with the following:

TABLE 1
FC3 Relaxer Composition

| | | | % |
|---|---|---|---|
| Trade Name: | INCI Name: | Supplier: | wt./wt. |
| Floraesters K-100 | Hydrolyzed Jojoba Esters | International | 33 |
| | (and) Jojoba Esters (and) | Flora | |
| | Water (Aqua) | Technologies | |
| Potassium Phosphate, | Potassium Phosphate | Sigma- | 13.6 |
| Tribasic Monohydrate | | Aldrich | |
| Deionized Water | Water | | 6.4 |
| KOH Solution | Potassium Hydroxide and | Mallinckrodt | 47 |
| (50% in water) | Water | Baker | |
| | | Total | 100 |

An 50% by weight potassium hydroxide solution was made by weighing approximately equal amounts of KOH and water in separate vessels. Water was added to the KOH. After cooling, the total weigh was adjusted by adding water to compensate for evaporative loss in the exothermic mixing process. A 47% weight equivalent amount of the 50% potassium hydroxide solution was combined with the remaining components to make the representative relaxer composition shown in Table 1. For example, about 47 g of the 50% potassium hydroxide solution was combined with the remaining components, the remaining components totaling about 53 g.

A potassium phosphate solution was then made with 6.4% wt. equivalents of water as measured by weight of the final relaxer composition. In a representative embodiment in accordance with the relaxer composition disclosed in Table 1, about 13.6 g of potassium phosphate was added to about 6.4 g of water for inclusion in a 100 g batch of relaxer composition.

Next, 33% wt. equivalents Floraesters K-100 as measured by weight of the final relaxer composition was mixed with the potassium phosphate solution at room temperature.

The KOH solution was slowly added to the mixture at room temperature with substantially continuous mixing until the batch was uniform and homogeneous.

Accordingly, a 100 g batch of relaxer composition, in accordance with the representative embodiment disclosed in Table 1, may comprise about 33 g Floraesters K-100, about 13.6 g potassium phosphate, about 6.4 g de-ionized water, and about 47 g 50% aqueous KOH solution.

Thereafter, the resulting composition (i.e., the “FC3” material) manufactured in accordance with a representative embodiment of the present invention may be incorporated into a substantially finished hair relaxer formula corresponding with, for example, either of the following:

TABLE 2
Representative Relaxer Formulation with 16.7% FC3

| | | | | % |
|---|---|---|---|---|
| Phase: | Trade Name: | INCI Name: | Supplier: | wt./wt. |
| A | Deionized | Water | | 43.80 |
| | Water | | | |
| | Veegum HS | Magnesium | RT Vanderbilt | 2.00 |
| | | Aluminum | | |
| | | Silicate | | |
| B | Glycerin, | Glycerin | Spectrum | 5.00 |
| | USP | | Chemical | |
| | Propylene | Propylene Glycol | Ashland Chemical | 5.00 |
| | Glycol | | Co. | |
| | FC3 | * not yet assigned | International Flora | 16.70 |
| | | | Technologies | |
| C | Merquat 10 | Polyquaternium- | Nalco Chemical | 15.00 |
| | (2%) | 10 | Company | |
| D | Lexemul 561 | Glyceryl Stearate | Inolex Chemicals | 5.00 |
| | | (and) PEG-100 | | |
| | | Stearate | | |
| | Lanette 16 | Cetyl Alcohol | Cognis Corp. | 2.50 |
| | Florasolvs | Jojoba Wax | International Flora | 2.00 |
| | PEG-120 | PEG-120 Esters | Technologies | |
| | Jojoba | | | |
| E | Benzyl | Benzyl Alcohol | VWR | 3.00 |
| | Alcohol | | | |
| | | | | 100.00 |

TABLE 3
Representative Relaxer Formulation with 25% FC3

| | | | | % |
|---|---|---|---|---|
| Phase | Trade Name | INCI Name | Supplier | wt./wt. |
| A | Deionized | Water | | 34.80 |
| | Water | | | |
| | Veegum HS | Magnesium | RT Vanderbilt | 2.20 |
| | | Aluminum | | |
| | | Silicate | | |
| B | Glycerin, USP | Glycerin | Spectrum | 3.00 |
| | | | Chemical | |
| | Propylene | Propylene Glycol | Ashland Chemical | 6.00 |
| | Glycol | | Co. | |
| | FC3 | * not yet assigned | International Flora | 25.00 |
| | | | Technologies | |
| C | Merquat 10 | Polyquaternium- | Nalco Chemical | 15.00 |
| | (2%) | 10 | Company | |
| D | Lexemul 561 | Glyceryl Stearate | Inolex Chemicals | 7.00 |
| | | (and) PEG-100 | | |
| | | Stearate | | |
| | Lanette 16 | Cetyl Alcohol | Cognis Corp. | 3.00 |
| | Florasolvs | Jojoba Wax | International Flora | 1.00 |
| | PEG-120 | PEG-120 Esters | Technologies | |
| | Jojoba | | | |
| E | Benzyl Alcohol | Benzyl Alcohol | VWR | 3.00 |
| | | | | 100.00 |

The pH of the 16.7% strength relaxer formulation in Table 2 was measured as 12.8 in an aqueous solution comprising 10% relaxer formulation and 90% water. The pH of the 25% strength relaxer formulation in Table 3 was measured as 13.1 in an aqueous solution comprising 10% relaxer formulation and 90% water.

To apply either of the exemplary relaxer formulations described in Table 2 or Table 3, the hair of a human subject is sectioned into four quadrants and clipped to keep each quadrant substantially separate. Quadrant-by-quadrant, the exemplary relaxer formulation is applied to the hair using a brush similar to the brash used by a cosmetologist to apply hair dye or other coloring agents to the hair. Within each quadrant, the relaxer formula is applied approximately one inch from the scalp in substantially horizontal segments. The relaxer formula is then brushed down the length of the hair to ensure saturation. Hair foils may be optionally employed to prevent or otherwise impede migration of the relaxer formula toward the subject's scalp.

The formulation is permitted to dwell on the hair, for example, for 15-30 minutes followed by rinse out with water. The hair is then shampooed using, for example, a neutralizing shampoo, and washed substantially free of relaxer formulation.

In the case where experimental trials may be needed to determine relative effectiveness of a given combination of relaxer compositions in accordance with various representative embodiments of the present invention, finished relaxer formulations, dwell times, hair types, and/or the like, etc., it may be beneficial to perform comparisons of results obtained from relaxer treatment of hair tresses rather than results obtained from treatments performed on the heads of human subjects. For example, the following protocol was used to determine relative relaxation performance of an exemplary embodiment of a relaxer composition (FC3) in a relaxer formulation (see, for example, Table 2 and Table 3) as a function of dwell time and percent (%) strength of the relaxer composition in the finished relaxer formulation.

Several one inch (1″), tight, curly, Caucasian hair tresses were washed with shampoo to remove any residue from the hair. 5-10 milliliters of finished relaxer formulation was applied to the hair tresses, combed through the hair, and manually manipulated for approximately 1-2 minutes using fingers and a comb. The hair tresses were then wrapped in foil with physical orientation of the hair fibers as straight as possible. The tresses were left for 15-60 minutes. Manipulation was repeated for approximately 1-2 minutes at the middle and end of the process. The hair tresses were then rinsed with water and dried. The hair tresses were then shampooed using a neutralizing shampoo and washed substantially free of relaxer formula.

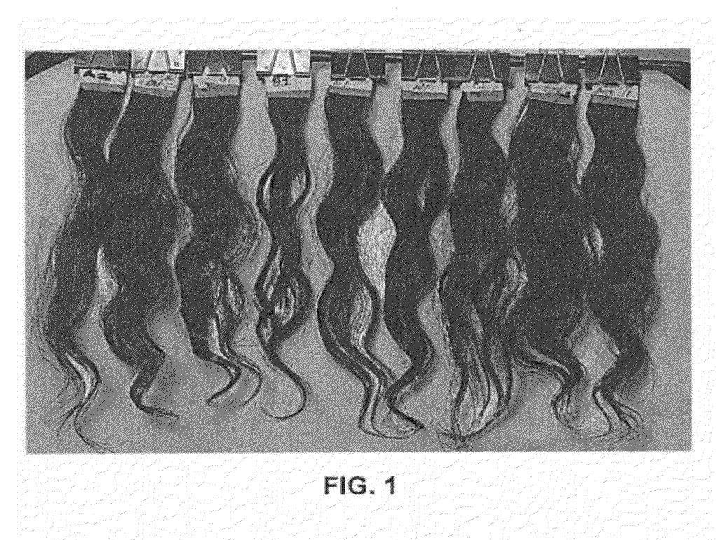

The following visual results were observed, in order from left to right, as depicted in FIG. 1: 50% strength FC3 for a dwell time of 30 minutes; 50% strength FC3 for a dwell time of 45 minutes; 50% strength FC3 for a dwell time of 60 minutes; 25% strength FC3 for a dwell time of 30 minutes; 25% strength FC3 for a dwell time of 45 minutes; 25% strength FC3 for a dwell time of 60 minutes; 10% strength FC3 for a dwell time of 30 minutes; 10% strength FC3 for a dwell time of 45 minutes; and 10% strength FC3 for a dwell time of 60 minutes.

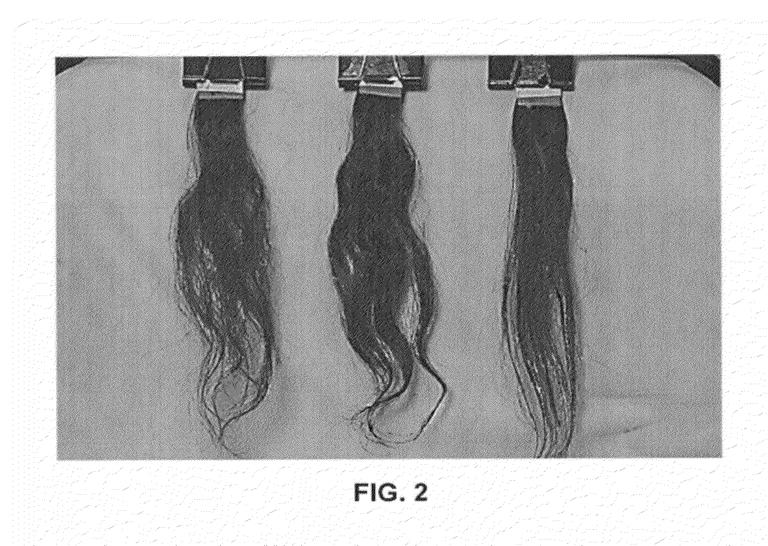

The following visual results were observed, in order from left to right, as depicted in FIG. 2: 10% strength FC3 for a dwell time of 30 minutes; 25% strength FC3 for a dwell time of 30 minutes; and 50% strength FC3 for a dwell time of 30 minutes.

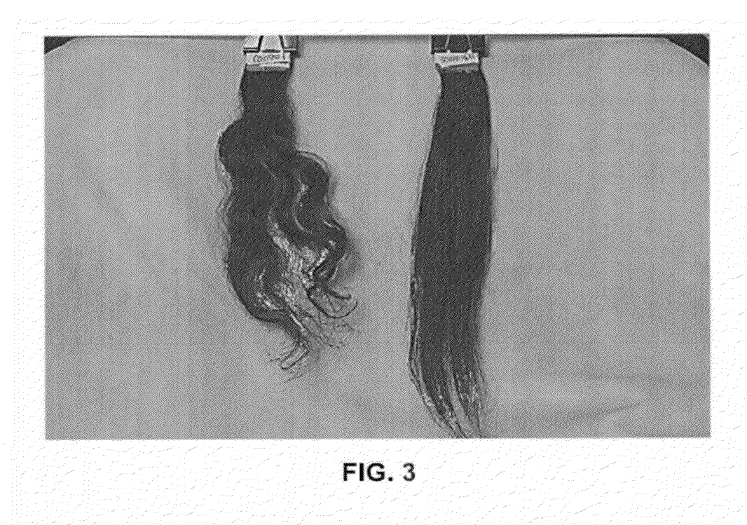

FIG. 3 shows the visual results of an untreated control hair tress on the left and a hair tress that was treated with 100% strength FC3 (i.e., no other relaxer formulation ingredients) for a dwell time of 30 minutes.

Table 4 shows relative relaxation measurements for various effective concentrations of exemplary relaxer compositions as a function of dwell times in accordance with various representative embodiments of the present invention.

TABLE 4
Relative Relaxation for Representative Relaxer Compositions

| | Dwell | |
|---|---|---|
| Relaxer Composition: | Time: | Relative Relaxation: |
| 6% KOH and 8.25% Floraesters K-100 | 30 min. | 100% Straight |
| 3% KOH and 4.1% Floraesters K-100 | 30 min. | 100% Straight |
| 1.5% KOH and 8.25% Floraesters K-100 | 30 min. | 40-50% Straight |
| 1.5% KOH and 8.25% Floraesters K-100 | 45 min. | 40-50% Straight |
| 1.5% KOH and 8.25% Floraesters K-100 | 60 min. | 40-50% Straight |
| 0.6% KOH and 8.25% Floraesters K-100 | 30 min. | 50-80% Straight |
| 0.6% KOH and 8.25% Floraesters K-100 | 45 min. | 50-60% Straight |
| 0.6% KOH and 8.25% Floraesters K-100 | 60 min. | 60% Straight |
| 3% KOH and 8.25% Floraesters K-100 | 30 min. | 65-100% Straight |
| 3% KOH and 8.25% Floraesters K-100 | 45 min. | 65-75% Straight |
| 3% KOH and 8.25% Floraesters K-100 | 60 min. | 65% Straight |
| 3.5% KOH and 8.25% Floraesters K-100 | 30 min. | 85% Straight |
| 4% KOH and 8.25% Floraesters K-100 | 30 min. | 85% Straight |
| 5% KOH and 8.25% Floraesters K-100 | 30 min. | 85% Straight |

As used herein, the word “exemplary” means “serving as an example, instance, or illustration”. “Exemplary” embodiments are not intended as models to be literally duplicated, but rather as examples that provide instances of embodiments that may be modified or altered in any way to create other embodiments. Any implementation described herein as “exemplary” is not necessarily to be construed as preferred or advantageous over other implementations.

While the foregoing detailed description will provide those skilled in the art with a convenient roadmap for implementing various embodiments of the invention, it should be appreciated that the particular embodiments described herein are only examples, and are not intended to limit the scope, applicability or configuration of the invention in any way. Various changes may be made in the function and arrangement of elements, components or steps herein described without departing from the scope of the invention and its legal equivalents.

What is claimed is:

1. A composition comprising:
(a) hydrolysate of an oil and/or wax, said hydrolysate comprising about 6-90% by weight of organic unsaponifiable materials and about 5-95% by weight of organic saponifiable materials;
(b) alcohol; and
(c) buffer substance;
the composition may optionally further comprise:
(d) water,
wherein said composition has a pH ranging from about 10 to about 14 when measured in an aqueous solution of 10% by weight of the composition and 90% by weight water.

2. The composition of claim 1, wherein the hydrolysate corresponds to the product produced by the hydrolysis of:
(a) amaranth seed oil, (b) anise seed oil, (c) avocado seed oil, (d) barley oil, (e) briza oil, (f) buck wheat oil, (g) candelilla wax, (h) carnuba wax, (i) cassia occidentalis oil, (j) coffee bean oil, (k) de-oiled lecithin, (l) dog fish oil, (m) esparto wax, (n) a fungal oil, (o) a microorganism oil, (p) guayule (plant material extract), (q) jojoba oil, (r) jurinea oil, (s) lanolin, (t) laurel berry oil, (u) olive oil concentrate (phytosqualene), (v) olive seed oil, (w) orange roughy oil, (x) ouricury wax, (y) quinoa seed oil, (z) rye germ oil, (aa) shark liver oil, (bb) shea butter, (cc) sperm whale oil, (dd) sugar cane wax, (ee) sunflower wax, (ff) tall oil, (gg) a tall oil distillate, (hh) wheat germ oil, (ii) rice bran wax, (jj) jojoba ester, (kk) sunflower oil, (ll) mango kernel oil, (mm) mango butter, (nn) moringa oil, (oo) moringa butter, (pp) an oil comprising sucrose bonded to 6, 7, or 8 fatty acids wherein each fatty acid is individually a saturated fatty acid, an unsaturated fatty acid, or a polyunsaturated fatty acid, (qq) algae oil, (rr) any derivative thereof, or (ss) any combination thereof.

3. The composition of claim 1, wherein the alcohol is:
(a) an alkyl alcohol, (b) a cycloalkyl alcohol, (c) a heterocycloalkyl alcohol, (d) an aryl alcohol, (e) a heteroaryl alcohol, (f) an arylalkyl alcohol, (g) a heteroarylalkyl alcohol, (h) an cycloalkylalkyl alcohol, (i) a diol, (j) a triol, (k) a bicyclic alcohol, (l) a tricyclic alcohol, (m) a tetracyclic alcohol, (n) a bicyclic alkyl alcohol, (o) a tricyclic alkyl alcohol, (p) a tetracyclic alkyl alcohol, (q) any derivative thereof, or (r) any combination thereof.

4. The composition of claim 1, wherein the alcohol is:
(a) methanol, (b) ethanol, (c) iso-propanol, (d) n-propanol, (e) n-butanol, (f) iso-butanol, (g) sec-butanol, (h) tert-butanol, (i) n-pentanol, (j) n-hexanol, (k) phenol, (l) benzyl alcohol, (m) 1-phenyl ethanol, (n) 1-phenyl-1-methyl ethanol, (o) 2-phenyl ethanol, (p) 3-phenyl propanol, (q) 2-phenyl propanol, (r) 1-phenyl propanol, (s) 1-phenyl-2-propanol, (t) cyclooctanol, (u) cycloheptanol, (v) cyclohexanol, (w) cyclopentanol, (x) cyclobutanol, (y) cyclopropanol, (z) cyclooctyl methyl alcohol, (aa) cycloheptyl methyl alcohol, (bb) cyclohexyl methyl alcohol, (cc) cyclopentyl methyl alcohol, (dd) cyclobutyl methyl alcohol, (ee) cyclopropyl methyl alcohol, (ff) 2-cyclooctyl ethanol, (gg) 2-cycloheptyl ethanol, (hh) 2-cyclohexyl ethanol, (ii) 2-cyclopentyl ethanol, (jj) 2-cyclobutyl ethanol, (kk) 2-cyclopropyl ethanol, (11) 1-cyclooctyl ethanol, (mm) 1-cycloheptyl ethanol, (nn) 1-cyclohexyl ethanol, (oo) 1-cyclopentyl ethanol, (pp) 1-cyclobutyl ethanol, (qq) 1-cyclopropyl ethanol, (rr) 1-cyclooctyl-1-methyl ethanol, (ss) 1-cycloheptyl-1-methyl ethanol, (tt) 1-cyclohexyl-1-methyl ethanol, (uu) 1-cyclopentyl-1-methyl ethanol, (vv) 1-cyclobutyl-1-methyl ethanol, (ww) 1-cyclopropyl-1-methyl ethanol, (xx) 3-cyclooctyl propanol, (yy) 3-cycloheptyl propanol, (zz) 3-cyclohexyl propanol, (aaa) 3-cyclopentyl propanol, (bbb) 3-cyclobutyl propanol, (ccc) 3-cyclopropyl propanol, (ddd) 2-cyclooctyl propanol, (eee) 2-cycloheptyl propanol, (fff) 2-cyclohexyl propanol, (ggg) 2-cyclopentyl propanol, (hhh) 2-cyclobutyl propanol, (iii) 2-cyclopropyl propanol, (jjj) 1-cyclooctyl propanol, (kkk) 1-cycloheptyl propanol, (lll) 1-cyclohexyl propanol, (mmm) 1-cyclopentyl propanol, (nnn) 1-cyclobutyl propanol, (ooo) 1-cyclopropyl propanol, (ppp) 1,2-propane diol, (qqq) 1,3-propane diol, (rrr) glycerol, (sss) a monoglyceride, (ttt) a diglyceride, (uuu) cedrenol, (vvv) cedrol, (www) jojoba alcohol, (xxx) borneol, (yyy) iso-borneol, (zzz) dodecanol, (aaaa) iso-dodecanol, (bbbb) icosanol, (cccc) iso-icosanol, (dddd) the alcohol portion of any wax/oil hydrolysate, (eeee) any derivative thereof, or (ffff) any combination thereof.

5. The composition of claim 1, wherein the oil and/or wax hydrolysate is formed from an oil and/or wax modified by at least one of:
(a) alkoxylation, (b) polymerization, (c) acetylation, (d) oxidation, (e) reduction, (f) concentration, (g) distillation, (h) hydrogenation, (i) partial hydrogenation, (j) interesterification, (k) double bond modification, (1) randomization, (m) refinement, (n) enzymatic treatment, (o) passive natural processes, (p) biological processes, (q) dilution, or (r) any combination thereof.

6. The composition of claim 1, wherein the buffer substance is a mixture of an acid and its conjugate base.

7. The composition of claim 1, wherein the buffer substance is:
(a) a zwitterion, (b) an addition salt of an organic molecule, (c) an organic acid, (d) an organic base, (e) an inorganic acid, (f) an inorganic base, (g) an amino acid, (h) an organic polymer, (i) an inorganic polymer, (j) a copolymer, (k) a block copolymer, (l) an inorganic salt, (m) an organic salt, (n) an oligomer comprising two to ten amino acids wherein the amino acids can be the same or different, (o) any derivative thereof, (p) an organic amine, or (q) any combination thereof

8. The composition of claim 1, wherein the buffer substance is: (a) mono potassium phosphate, (b) di potassium phosphate, (c) tri potassium phosphate, (d) mono sodium phosphate, (e) di sodium phosphate, (f) tri sodium phosphate, (g) mono lithium phosphate, (h) di lithium phosphate, (i) tri lithium phosphate, (j) calcium phosphate, (k) magnesium phosphate, (l) phosphoric acid, (m) polyphosphoric acid, (n) a polyethylene imine (PEI), (o) any derivative thereof, (p) any anhydrous form thereof, (q) any hydrated form thereof, or (r) any combination thereof.

9. The composition of claim 1, further comprising a hydroxide salt.

10. The composition of claim 9, wherein the hydroxide salt is:
(a) LiOH, (b) NaOH, (c) KOH, (d) Mg(OH)₂, (e) Ca(OH)₂, (f) Zn(OH)₂, (g) Fe(OH)₂, (h) Fe(OH)₃, (i) Mn(OH)₂, (j) ammonium hydroxide, (k) aluminum hydroxide, (l) hydroxyl amine, (k) any valence state variant thereof, or (k) any combination thereof.

11. The composition of claim 9, wherein the composition comprises:
(a) from about 0.5-7.5% by weight of the buffer substance; and
(b) from about 1-5% by weight of the alcohol;
wherein the percentage (%) by weight is based on the total weight of the composition; and
wherein the pH of the composition ranges from about 10 to about 12.

12. The composition of claim 9, wherein a ratio of the alcohol to a blend comprising: the water, the hydroxide salt, the buffer substance, and the hydrolysate; ranges from about 1:1 to about 1:10 on a weight-to-weight basis.

13. The composition of claim 12, wherein in the blend:
(a) the amount of water ranges from about 18-29% by weight based on the weight of the blend;
(b) the amount of hydroxide salt ranges from about 18-29% by weight based on the weight of the blend;
(c) the amount of hydrolysate ranges from about 10-40% by weight based on the weight of the blend; and
(d) the amount of buffer substance ranges from about 15-25% by weight based on the weight of the blend.

14. The composition of claim 12, wherein the alcohol is benzyl alcohol, the hydroxide salt is potassium hydroxide, the buffer substance is tri potassium phosphate, and the hydrolysate is jojoba oil hydrolysate.

15. The composition of claim 12, wherein the alcohol is 2-phenyl ethanol, the hydroxide salt is potassium hydroxide, the buffer substance is tri potassium phosphate, and the hydrolysate is jojoba oil hydrolysate.

16. The composition of claim 12, wherein the alcohol is 3-phenyl propanol, the hydroxide salt is potassium hydroxide, the buffer substance is tri potassium phosphate, and the hydrolysate is jojoba oil hydrolysate.

17. The composition of claim 12, wherein the alcohol is cedrol, the hydroxide salt is potassium hydroxide, the buffer substance is tri potassium phosphate mono hydrate, and the hydrolysate is jojoba oil hydrolysate.

18. The composition of claim 12, wherein the alcohol is benzyl alcohol, the hydroxide salt is potassium hydroxide, the buffer substance is a polyethylene imine (PEI), and the hydrolysate is jojoba oil hydrolysate.

19. The composition of claim 12, wherein the alcohol is 2-phenyl ethanol, the hydroxide salt is potassium hydroxide, the buffer substance is a polyethylene imine (PEI), and the hydrolysate is jojoba oil hydrolysate.

20. The composition of claim 12, wherein the alcohol is 3-phenyl propanol, the hydroxide salt is potassium hydroxide, the buffer substance is a polyethylene imine (PEI), and the hydrolysate is jojoba oil hydrolysate.

21. The composition of claim 12, wherein the alcohol is cedrol, the hydroxide salt is potassium hydroxide, the buffer substance is a polyethylene imine (PEI), and the hydrolysate is jojoba oil hydrolysate.

22. The composition of claim 12, wherein a ratio of the alcohol to the blend on a weight-to-weight basis is about 4:1.

23. A method of making a composition, comprising:
combining water, an alcohol, a buffer substance, and an oil and/or wax hydrolysate, to form a mixture,
wherein the hydrolysate is a hydrolysate of an oil and/or wax comprising about 6-95% by weight of organic unsaponifiable materials and about 5-94% by weight of organic saponifiable materials, and
adjusting the pH of the mixture to give a pH range from about 10 to about 14 when measured in an aqueous solution of 10% by weight of the composition and 90% by weight water.

24. The method of claim 23, wherein the pH is adjusted by addition of an organic hydroxide salt and/or inorganic hydroxide salt to the mixture.

25. A composition produced by the method of claim 23.

26. A method of straightening or relaxing hair, comprising:
applying the composition of claim 1 to the hair in an amount sufficient to relax the hair.

27. A method of straightening or relaxing hair in need thereof, comprising:
applying the composition of claim 1 to the hair in an amount sufficient to relax the hair in need thereof

28. The method of claim 26, wherein the hair is human hair.

29. A kit comprising the composition of claim 1.

30. The kit of claim 29, further comprising instructions for applying the composition to hair.

31. Hair that has been straightened or relaxed by the composition of claim 1.

32. Hair that has been straightened or relaxed by the method of claim 26.
